(19) United States
(12) Patent Application Publication
(10) Pub. No.: US 2008/0057528 A1
(43) Pub. Date: Mar. 6, 2008

(54) Detection of hydrogen peroxide released by enzyme-catalyzed oxidation of an analyte

(75) Inventors: Curtis Sayre, Atlanta, GA (US); Shawn R. Feaster, Duluth, GA (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 11/513,898

(22) Filed: Aug. 30, 2006

(51) Int. Cl. C12Q 1/30

(52) U.S. Cl. 435/27

(57) ABSTRACT

A diagnostic test kit for detecting hydrogen peroxide released by enzyme-catalyzed oxidation of an analyte within a test sample is provided. The test kit comprises a lateral flow device that contains a chromatographic medium, wherein the chromatographic medium defines a detection zone within which a chromogen is substantially non-diffusively immobilized in conjunction with an anionic compound. The chromogen is a leuco base, or a derivative thereof, which is capable of undergoing a detectable color change upon oxidation.

### BACKGROUND OF THE INVENTION

Hydrogen peroxide is produced in enzyme-catalyzed reactions of various chemical or biological substances (analytes), such as glucose, cholesterol, uric acid, triglycerides, creatine kinase, creatinine, etc. The quantity of the analyte within a given test sample may be determined from the amount of hydrogen peroxide produced. Diabetes, for instance, is often diagnosed by detecting the presence of glucose in urine. For example, a urine test sample may be contacted with a glucose oxidase enzyme, which catalyzes the aerobic oxidation of glucose into gluconic acid and hydrogen peroxide. Indicators (e.g., leuco dyes) are typically employed that undergo a color change in the presence of hydrogen peroxide. The color change produced is indicative of the amount of H₂O₂ present, as well as the analyte content of the fluid being tested. Unfortunately, however, many of the mechanisms for performing such a test are overly complex, expensive, and consuming.

As such, a need currently exists for an improved technique for detecting hydrogen peroxide generated by enzyme-catalyzed oxidation of an analyte.

### SUMMARY OF THE INVENTION

In accordance with one embodiment of the present invention, a diagnostic test kit is disclosed for detecting hydrogen peroxide released by enzyme-catalyzed oxidation of an analyte. The test kit comprises a lateral flow device that contains a chromatographic medium. The chromatographic medium defines a detection zone within which a chromogen is substantially non-diffusively immobilized in conjunction with an anionic compound. The chromogen is a leuco base, or a derivative thereof, which is capable of undergoing a detectable color change upon oxidation.

In accordance with another embodiment of the present invention, a method is disclosed for detecting hydrogen peroxide released by enzyme-catalyzed oxidation of an analyte. The method comprises providing a lateral flow device that comprises-a chromatographic medium, the chromatographic medium defining a detection zone within which an oxidizable chromogen is substantially non-diffusively immobilized in conjunction with an anionic compound. The oxidizable chromogen is a leuco base or a derivative thereof. The hydrogen peroxide is reacted with an electron donor to form an intermediate compound. The intermediate compound is allowed to flow through the chromatographic medium and contact the detection zone, whereby the intermediate compound oxidizes the chromogen. The oxidized chromogen has a color that differs from the color of the oxidizable chromogen. The color of the oxidized chromogen is detected.

Other features and aspects of the present invention are discussed in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figure in which:

FIG. 1 is a perspective view of one embodiment of a lateral flow device that may be used in the present invention.

Repeat use of reference characters in the present specification and drawing is intended to represent same or analogous features or elements of the invention.

### DETAILED DESCRIPTION OF REPRESENTATIVE EMBODIMENTS

### DEFINITIONS

As used herein, the term “test sample” generally refers to any material suspected of containing an analyte. The test sample may be derived from any biological source, such as a physiological fluid, including, blood, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid, semen, and so forth. Besides physiological fluids, other liquid samples may be used such as water, food products, and so forth, for the performance of environmental or food production assays. In addition, a solid material suspected of containing the analyte may be used as the test sample. The test sample may be used directly as obtained from the biological source or following a pretreatment to modify the character of the sample. For example, such pretreatment may include preparing plasma from blood, diluting viscous fluids, and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the analyte.

### DETAILED DESCRIPTION

Reference now will be made in detail to various embodiments of the invention, one or more examples of which are set forth below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention covers such modifications and variations as come within the scope of the appended claims and their equivalents.

Generally speaking, the present invention is directed to a diagnostic test kit that is configured to detect the presence hydrogen peroxide released by enzyme-catalyzed oxidation of an analyte, such as glucose, galactose, monoamine, L-amino acid, alcohol, xanthine, cholesterol, lactate, uric acid, triglycerides, creatine kinase, creatinine, or sarcosine. Any of a variety of enzymes may be employed for catalyzing the oxidation reaction, including for instance, oxidases, such as galactose oxidase, glucose oxidase, cholesterol oxidase, amine oxidase, various amino acid oxidases, polyphenol oxidase, xanthine oxidase, uricase, etc.; dehydrogenases, such as alcohol dehydrogenase, lactate dehydrogenase, malate dehydrogenase, glyceraldehyde-3-phosphate dehydrogenase, glycerol dehydrogenase, glucose-6-phosphate dehydrogenase, etc.; lipases, such as lipoprotein lipase, phospholipase, etc.; esterases, such as cholesterol esterase, cholinesterase, etc. Glucose, for instance, may be oxidized in the presence of glucose oxidase to yield gluconic acid and hydrogen peroxide. The enzyme may be used alone or as a conjugate to a biological molecule or a member of a specific binding pair.

Regardless of the manner in which the enzyme-catalyzed reaction is conducted, hydrogen peroxide is released through oxidation of the analyte. The amount of hydrogen peroxide released is generally proportional to the amount of the analyte within the test sample. Thus, the presence or concentration of the analyte may be determined by directly or indirectly detecting the presence of the released hydrogen peroxide. More specifically, the diagnostic kit of the present invention employs a chromogen that is capable of undergoing a detectable color change in the presence of hydrogen peroxide or another oxidizing agent. Without intending to be limited by theory, it is believed that oxidation of the chromogen induces either a shift of the absorption maxima towards the red end of the spectrum (“bathochromic shift”) or towards the blue end of the spectrum (“hypsochromic shift”). The absorption shift provides a color difference that is detectable, either visually or through instrumentation, to indicate the presence of hydrogen peroxide within the test sample. For example, prior to contact with a test sample, the chromogen may be colorless or it may possess a certain color. However, after contacting the test sample and reacting with hydrogen peroxide released by enzyme-catalyzed oxidation of the analyte, the chromogen exhibits a color that is different than its initial color. The color change may thus be readily correlated to the presence of the analyte (e.g., cholesterol, glucose, etc.) in the test sample.

The chromogen of the present invention is a leuco base, or a derivative thereof, which is capable of exhibiting a detectable change in color upon oxidation. For example, arylmethane leuco bases (e.g., diarylmethanes and triarylmethanes) are particularly suitable oxidizable chromogens for use in the present invention. Triarylmethane leuco bases, for example, have the following general structure:

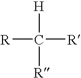

wherein R, R′, and R″ are independently selected from substituted and unsubstituted aryl groups, such as phenyl, naphthyl, anthracenyl, etc. The aryl groups may be substituted with functional groups, such as amino, hydroxyl, carbonyl, carboxyl, sulfonic, alkyl, and/or other known functional groups. Examples of such triarylmethane leuco bases include leucomalachite green, pararosaniline base, crystal violet lactone, crystal violet leuco, crystal violet, Cl Basic Violet 1, Cl Basic Violet 2, Cl Basic Blue, Cl Victoria Blue, N-benzoyl leuco-methylene, etc. Likewise suitable diarylmethane leuco bases may include 4,4′-bis (dimethylamino) benzhydrol (also known as “Michler's hydrol”), Michler's hydrol leucobenzotriazole, Michler's hydrol leucomorpholine, Michler's hydrol leucobenzenesulfonamide, etc. In addition to arylmethane leuco bases, other chromogens that may exhibit a detectable color change in the presence of hydrogen peroxide or another oxidizing agent are described in U.S. Pat. No. 4,089,747 to Bruschi, which is incorporated herein in its entirety by reference thereto for all purposes.

In one particular embodiment, the chromogen is leucomalachite green (or an analog thereof), which is generally colorless and has the following structure:

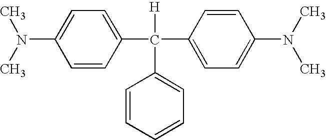

Upon oxidation with hydrogen peroxide, leucomalachite green forms malachite green carbinol (Solvent Green 1), which has the following structure:

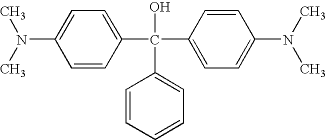

The carbinol form of leucomalachite green is also colorless. However, under acidic conditions, one or more free amino groups of the leucomalachite green carbinol form may be protonated to form malachite green (also known as aniline green, basic green 4, diamond green B, or victoria green B), which is green in color and has the following structure:

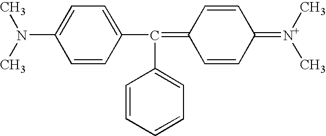

The hydrogen peroxide released by the enzyme-catalyzed oxidation of the analyte may directly induce a color change in the chromogen as described above. Because hydrogen peroxide has a relatively low oxidation potential for certain chromogens, however, it is sometimes difficult to detect the color change (e.g., visibly) when the peroxide is released in low concentrations (e.g., less than 5 wt. % of the test sample). In this regard, an electron donor may optionally be employed to react with hydrogen peroxide and produce an intermediate compound having a higher oxidation potential for the chromogen than hydrogen peroxide. A variety of known electron donors may be employed for this purpose. In one embodiment, for example, an excess amount of iodide ions (I⁻) in aqueous solution may react with hydrogen peroxide to form triiodide ions (I₃⁻), which have a much greater oxidation potential than hydrogen peroxide. Exemplary sources of ionic iodide include hydrogen iodide (HI) and water-soluble iodide salts, such as alkali metal iodide salts (e.g., potassium iodide (KI), sodium iodide (NaI), lithium iodide), ammonium iodide (NH₄I), calcium iodide (CaI₂), etc.). Other suitable electron donors may include a source of thiocyanate ions, such as sodium thiocyanate, potassium thiocyanate, ammonium thiocyanate, and other thiocyanate salts. Metals, such as iron(II), may also be used as electron donors. For example, Fenton's reagent is a solution that is formed by reaction of iron(II) and hydrogen peroxide. That is, iron(II) is oxidized to iron(III) by hydrogen peroxide to form a hydroxyl radical and a hydroxyl anion. Iron(III) is then reduced back to iron(II) by the same hydrogen peroxide to a peroxide radical and a proton. The resulting reagent has a strong oxidation potential for the chromogen. Still other suitable electron donors are described in U.S. Patent Application Publication No. 2002/0119136 to Johansen, which is incorporated herein in its entirety by reference thereto for all purposes.

Although the electron donor may provide intermediate compounds with a high oxidation potential, the concentration of such compounds may nevertheless be too low in some cases to produce the desired color change in the chromogen. For example, high concentrations of a triiodide ion may result in a color (e.g., golden brown) that is visible to the human eye. However, as its concentration decreases, the color becomes less apparent. Thus, a color developer may be employed that complexes to the intermediate compound (e.g., triiodide ions) to form a more intense color. One particular example of such a color developer is starch, which encompasses both natural starch and modified derivatives, such as dextrinated, hydrolyzed, alkylated, hydroxyalkylated, acetylated or fractionated starch. Starches are generally formed from two structurally distinctive polysaccharides, i.e., α-amylose and amylopectin, both of which are comprised of α-D-glucopyranose units. The starches may be of or derived from any origin, such as corn starch, wheat starch, potato starch, tapioca starch, sago starch, rice starch, waxy corn starch or high amylose corn starch. When employed in conjunction with an iodide source, such as described above, the a-amylose portion of the starch may entrap or bind to the triiodide ion to form a linear triiodide ion complex that is water-soluble and has an intense blue color.

The extent to which the electron donor and/or color developer facilitate the desired color change depends in part on their concentration. That is, too large of a concentration of one or more of these components may overwhelm the chromogen and stifle the oxidation reaction. On the other hand, too low of a concentration may not enhance the oxidation potential to the desired extent. In this regard, the electron donor (e.g., iodide source) may be employed in an amount from about 0.01 to about 2000 millimoles (“mM”), in some embodiments from about 0.1 to about 1000 mM, and in some embodiments, from about 1 to about 100 mM per liter of the test sample. The color developer (e.g., starch) may likewise be employed in an amount from about 0.001 to about 10 wt. %, in some embodiments from about 0.01 to about 5 wt. %, and in some embodiments, from about 0.1 to about 2 wt. % based on the weight of the test sample.

To achieve the desired color change in accordance with the present invention, the chromogen is applied to a reaction medium in a manner so that it does not substantially diffuse through the matrix of the medium (i.e., non-diffusively immobilized). This enables a user to readily detect the change in color that occurs upon oxidation of the chromogen. For example, a solution containing the chromogen may be initially applied to the reaction medium within a detection zone. The chromogenic solution may contain an aqueous and/or non-aqueous solvent depending on the material used to form the chromatographic medium. Suitable non-aqueous solvents may include glycols (e.g., propylene glycol, butylene glycol, triethylene glycol, hexylene glycol, polyethylene glycols, ethoxydiglycol, and dipropyleneglycol); alcohols (e.g., methanol, ethanol, n-propanol, and isopropanol); triglycerides; ethyl acetate; acetone; triacetin; acetonitrile, tetrahydrafuran; xylenes; formaldehydes (e.g., dimethylformamide); etc. The amount of the solvent and chromogen in the solution may generally vary based on the desired level of sensitivity. For instance, in some embodiments, the chromogen may be present at a concentration from about 0.1 to about 100 milligrams per milliliter of solvent, in some embodiments from about 0.5 to about 60 milligrams per milliliter of solvent, and in some embodiments, from about 1 to about 40 milligrams per milliliter of solvent.

Regardless, the solution may be dried to remove the solvent and leave a residue of the chromogen on the medium. The chromogen will generally remain within the detection zone until contacted with the fluidic test sample. Because the chromogen is water-soluble, however, it would normally dissolve and flow with the test sample unless otherwise immobilized. Thus, in accordance with the present invention, the chromogen is substantially non-diffusively immobilized within the detection zone in conjunction with an anionic compound, i.e., a compound that contains one or more anions or is capable of forming one or more ions in solution. Such anionic compounds may facilitate immobilization of the chromogen in a variety of ways. For example, anionic compounds may also enhance the charge of the chromogen so that forms an ionic bond with one or more functional groups present on the surface of the chromatographic medium. In addition, certain anionic compounds (e.g., acids) may form a substantially water-insoluble precipitate when reacted with a leuco base or derivative thereof (e.g., protonated leuco base). Of course, the anionic compound may also provide a variety of other benefits. For example, a small amount of the chromogen may undergo an oxidation reaction if left in air or other oxidizing environment for too great a period of time. This may lead to a change in color that would indicate a “false positive” or at the very least, adversely affect the ability to semi-quantitatively or quantitatively determine the presence of the analyte. The anionic compound may help protect the chromogen from inadvertent oxidation and thus reduce “false positives.”

The selection of the anionic compound depends on a variety of factors, including the nature of the chromogen and its concentration. Suitable anionic compounds for use in the present invention may include, for instance, inorganic acids, such as hydrochloric acid, nitric acid, sulfuric acid, phosphoric acid, polyphophoric acid, boric acid, boronic acid, etc.; organic acids, including carboxylic acids, such as acrylic acid, methacrylic acid, malonic acid, succinic acid, salicylic acid, sulfosalicylic acid, adipic acid, maleic acid, malic acid, oleic acid, gallic acid, tartaric acid, citric acid, formic acid, acetic acid, glycolic acid, oxalic acid, propionic acid, phthalic acid, isophthalic acid, glutaric acid, gluconic acid, lactic acid, aspartic acid, glutaminic acid, itaconic acid, trifluoroacetic acid, barbituric acid, cinnamic acid, benzoic acid, 4-hydroxybenzoic acid, aminobenzoic acid, etc.; sulfonic acids, such as methanesulfonic acid, benzenesulfonic acid, toluenesulfonic acid, trifluoromethanesulfonic acid, styrenesulfonic acid, naphthalene disulfonic acid, hydroxybenzenesulfonic acid, etc.; polymeric acids, such as poly(acrylic) or poly(methacrylic) acid and copolymers thereof (e.g., maleic-acrylic, sulfonic-acrylic, and styrene-acrylic copolymers), carageenic acid, carboxymethyl cellulose, alginic acid, etc.; and so forth. Anhydrides (e.g., maleic anhydride) and salts of the aforementioned acids may also be employed. The salts may be in the form of metal salts, such as sodium salts, potassium salts, calcium salts, cesium salts, zinc salts, copper salts, iron salts, aluminum salts, zirconium salts, lanthanum salts, yttrium salts, magnesium salts, strontium salts, cerium salts), or salts prepared by reacting the acids with amines (e.g., ammonia, triethylamine, tributyl amine, piperazine, 2-methylpiperazine, polyallylamine).

The degree to which the chromogen is immobilized may depend on the concentration of the anionic compound. For instance, the concentration of the anionic compound in the chromogenic solution may be from about 0.1 to about 20 millimoles per liter (“mM”), in some embodiments from about 1 mM to about 10 mM, and in some embodiments, from about 2 mM to about 8 mM.

In accordance with the present invention, the desired reaction time between the reagents (e.g., enzyme, peroxide, electron donor, color developer, etc.) may be achieved by selectively controlling the medium in which the reactions occur. That is, the reaction medium is chromatographic in nature so that the hydrogen peroxide and/or other reagents are allowed to flow laterally in a consistent and controllable manner. While laterally flowing through the medium, the hydrogen peroxide oxidizes the chromogen, which is contained within a discrete detection zone for analysis. Due to the nature of the controlled fluid flow, any unreacted reagents travel to the end of the reaction medium so that it is unable to adversely interfere with observance of the chromogen within the detection zone.

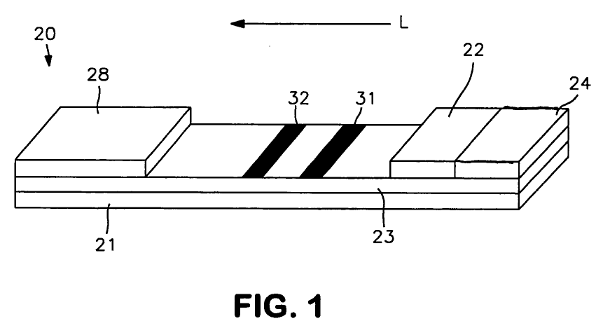

In this regard, FIG. 1 illustrates one particular embodiment of a lateral flow device 20 for detecting the presence of an analyte in accordance with the present invention. As shown, the lateral flow device 20 contains a chromatographic medium 23 optionally supported by a rigid support material 21. The chromatographic medium 23 may be made from any of a variety of materials through which the test sample is capable of passing. For example, the chromatographic medium 23 may be a porous membrane formed from synthetic or naturally occurring materials, such as polysaccharides (e.g., cellulose materials such as paper and cellulose derivatives, such as cellulose acetate and nitrocellulose); polyether sulfone; polyethylene; nylon; polyvinylidene fluoride (PVDF); polyester; polypropylene; silica; inorganic materials, such as deactivated alumina, diatomaceous earth, MgSO₄, or other inorganic finely divided material uniformly dispersed in a porous polymer matrix, with polymers such as vinyl chloride, vinyl chloride-propylene copolymer, and vinyl chloride-vinyl acetate copolymer; cloth, both naturally occurring (e.g., cotton) and synthetic (e.g., nylon or rayon); porous gels, such as silica gel, agarose, dextran, and gelatin; polymeric films, such as polyacrylamide; and so forth. In one particular embodiment, the chromatographic medium 23 is formed from nitrocellulose and/or polyether sulfone materials. It should be understood that the term “nitrocellulose” refers to nitric acid esters of cellulose, which may be nitrocellulose alone, or a mixed ester of nitric acid and other acids, such as aliphatic carboxylic acids having from 1 to 7 carbon atoms.

The size and shape of the chromatographic medium 23 may generally vary as is readily recognized by those skilled in the art. For instance, a porous membrane strip may have a length of from about 10 to about 100 millimeters, in some embodiments from about 20 to about 80 millimeters, and in some embodiments, from about 40 to about 60 millimeters. The width of the membrane strip may also range from about 0.5 to about 20 millimeters, in some embodiments from about 1 to about 15 millimeters, and in some embodiments, from about 2 to about 10 millimeters. Likewise, the thickness of the membrane strip is generally small enough to allow transmission-based detection. For example, the membrane strip may have a thickness less than about 500 micrometers, in some embodiments less than about 250 micrometers, and in some embodiments, less than about 150 micrometers.

As stated above, the support 21 carries the chromatographic medium 23. For example, the support 21 may be positioned directly adjacent to the chromatographic medium 23 as shown in FIG. 1, or one or more intervening layers may be positioned between the chromatographic medium 23 and the support 21. Regardless, the support 21 may generally be formed from any material able to carry the chromatographic medium 23. The support 21 may be formed from a material that is transmissive to light, such as transparent or optically diffuse (e.g., transluscent) materials. Also, it is generally desired that the support 21 is liquid-impermeable so that fluid flowing through the medium 23 does not leak through the support 21. Examples of suitable materials for the support include, but are not limited to, glass; polymeric materials, such as polystyrene, polypropylene, polyester (e.g., Mylar® film), polybutadiene, polyvinylchloride, polyamide, polycarbonate, epoxides, methacrylates, and polymelamine; and so forth. To provide a sufficient structural backing for the chromatographic medium 23, the support 21 is generally selected to have a certain minimum thickness. Likewise, the thickness of the support 21 is typically not so large as to adversely affect its optical properties. Thus, for example, the support 21 may have a thickness that ranges from about 100 to about 5,000 micrometers, in some embodiments from about 150 to about 2,000 micrometers, and in some embodiments, from about 250 to about 1,000 micrometers. For instance, one suitable membrane strip having a thickness of about 125 micrometers may be obtained from Millipore Corp. of Bedford, Mass. under the name “SHF180UB25.”

As is well known the art, the chromatographic medium 23 may be cast onto the support 21, wherein the resulting laminate may be die-cut to the desired size and shape. Alternatively, the chromatographic medium 23 may simply be laminated to the support 21 with, for example, an adhesive. In some embodiments, a nitrocellulose or nylon porous membrane is adhered to a Mylar® film. An adhesive is used to bind the porous membrane to the Mylar® film, such as a pressure-sensitive adhesive. Laminate structures of this type are believed to be commercially available from Millipore Corp. of Bedford, Mass. Still other examples of suitable laminate device structures are described in U.S. Pat. No. 5,075,077 to Durley. III, et al., which is incorporated herein in its entirety by reference thereto for all purposes.

The device 20 may also contain an absorbent material 28 that is positioned adjacent to the medium 23. The absorbent material 28 assists in promoting capillary action and fluid flow through the medium 23. In addition, the absorbent material 28 receives fluid that has migrated through the entire chromatographic medium 23 and thus draws any unreacted components away from the detection region. Some suitable absorbent materials that may be used in the present invention include, but are not limited to, nitrocellulose, cellulosic materials, porous polyethylene pads, glass fiber filter paper, and so forth. The absorbent material may be wet or dry prior to being incorporated into the device. Pre-wetting may facilitate capillary flow for some fluids, but is not typically required. Also, as is well known in the art, the absorbent material may be treated with a surfactant to assist the wicking process.

To initiate the assay, a user may directly apply the test sample to a portion of the chromatographic medium 23 through which it may then travel in the direction illustrated by arrow “L” in FIG. 1. Alternatively, the test sample may first be applied to a sample application zone 24 that is in fluid communication with the chromatographic medium 23. The sample application zone 24 may be formed on the medium 23. Alternatively, as shown in FIG. 1, the sample application zone 24 may be formed by a separate material, such as a pad. Some suitable materials that may be used to form such sample pads include, but are not limited to, nitrocellulose, cellulose, porous polyethylene pads, and glass fiber filter paper. If desired, the sample application zone 24 may also contain one or more pretreatment reagents, either diffusively or non-diffusively attached thereto.

To facilitate detection of the analyte in the manner described above, various reagents are employed, such as enzymes, electron donors, color developers, etc. In some embodiments, one or more of the reagents may be mixed with the test sample prior to application to the device 20. For example, the test sample may be allowed to incubate with the enzyme for a certain period of time. Those skilled in the art readily recognize that the time of incubation for an enzyme-catalyzed reaction depends on the activity of the enzyme of interest, which in turn depends on in part on the temperature, pH, substrate concentration, the presence of inhibitors (competitive (binds to enzyme), uncompetitive (binds to enzyme-substrate complex), or noncompetitive (binds to enzyme and/or enzyme-substrate complex)), and so forth. These factors may be selectively controlled as desired to increase or decrease the incubation time. For example, the time for incubation may be greater than about 1 minute, in some embodiments from about 5 to about 50 minutes, and in some embodiments, from about 10 to about 25 minutes. Likewise, the pH may be selectively controlled to facilitate enzyme activity. For example, high levels of basic substances (e.g., amines) within a test sample may result in a pH that is too high for optimum activity of some enzymes, e.g., greater than 8. Specifically, an enzyme may possess optimum activity at a pH level of from about 3 to about 8, and in some embodiments, from about 4 to about 7. Thus, if desired, a buffer or other pH-altering compound may be employed to maintain the desired pH. Some biologically compatible buffers that may be used to maintain the desired pH include borate buffers, phosphate-buffered saline (PBS), 2-(N-morpholino) ethane sulfonic acid (“MES”), tris-hydroxymethylaminomethane (“Tris”), citrate buffers, and so forth.

After incubation, any enzyme present within the test sample will typically oxidize the analyte and release hydrogen peroxide. The peroxide-containing test sample may then be applied to the assay device 20. Alternatively, the enzyme and/or other reagent(s) may be diffusively immobilized on the device 20 prior to application of the test sample. Such pre-application provides a variety of benefits, including the elimination of the need for a subsequent user to handle and mix the reagents with the test sample or a diluent. This is particularly useful in point-of-care applications when the user is not generally a trained lab technician or medical professional. The reagent(s) may be disposed upstream from, downstream from, or at the sample application zone 24. In this manner, the test sample is capable of mixing with the analyte upon application. When disposed downstream from the point where the test sample is to be applied, the test sample is capable of mixing with and dissolving or re-suspending the reagents upon application.

In the illustrated embodiment, for example, a reagent zone 22 is employed that is in fluid communication with the sample application zone 24. As shown in FIG. 1, the reagent zone 22 is formed from a separate material or pad. Such a reagent pad may be formed from any material through which the test sample is capable of passing, such as glass fibers. Alternatively, the reagent zone 22 may simply be formed on the medium 23. Regardless, the reagent zone 22 may be applied with one or more solutions containing reagents, such as enzymes, electron donors, color developers, etc. and dried. Thus, the test sample may contact the reagent zone 22 and to generate hydrogen peroxide or other intermediate compounds before reaching a detection zone 31, which is located downstream from the reagent zone 22 and immobilized the oxidizable chromogen described above.

One benefit of the lateral flow device of the present invention is its ability to readily incorporate one or more additional zones to facilitate analyte detection. For example, referring again to FIG. 1, a control zone 32 may also be employed in the lateral flow device 20 for improving detection accuracy. The control zone 32 gives a signal to the user that the test is performing properly. More specifically, control reagents may be employed that flow through the chromatographic medium 23 upon contact with a sufficient volume of the test sample. These control reagents may then be observed, either visually or with an instrument, within the control zone 32. The control reagents generally contain a detectable substance, such as luminescent compounds (e.g., fluorescent, phosphorescent, etc.); radioactive compounds; visual compounds (e.g., colored dye or metallic substance, such as gold); liposomes or other vesicles containing signal-producing substances; enzymes and/or substrates, and so forth. Other suitable detectable substances may be described in U.S. Pat. No. 5,670,381 to Jou. et al. and U.S. Pat. No. 5,252,459 to Tarcha, et al., which are incorporated herein in their entirety by reference thereto for all purposes.

If desired, the detectable substances may be disposed on particles (sometimes referred to as “beads” or “microbeads”). Among other things, the particles enhance the ability of the detectable substance to travel through a chromatographic medium. For instance, naturally occurring particles, such as nuclei, mycoplasma, plasmids, plastids, mammalian cells (e.g., erythrocyte ghosts), unicellular microorganisms (e.g., bacteria), polysaccharides (e.g., agarose), etc., may be used. Further, synthetic particles may also be utilized. For example, in one embodiment, latex microparticles that are labeled with a fluorescent or colored dye are utilized. Although any synthetic particle may be used in the present invention, the particles are typically formed from polystyrene, butadiene styrenes, styreneacrylic-vinyl terpolymer, polymethylmethacrylate, polyethylmethacrylate, styrene-maleic anhydride copolymer, polyvinyl acetate, polyvinylpyridine, polydivinylbenzene, polybutyleneterephthalate, acrylonitrile, vinylchloride-acrylates, and so forth, or an aldehyde, carboxyl, amino, hydroxyl, or hydrazide derivative thereof. When utilized, the shape of the particles may generally vary. In one particular embodiment, for instance, the particles are spherical in shape. However, it should be understood that other shapes are also contemplated by the present invention, such as plates, rods, discs, bars, tubes, irregular shapes, etc. In addition, the size of the particles may also vary. For instance, the average size (e.g., diameter) of the particles may range from about 0.1 nanometers to about 1,000 microns, in some embodiments, from about 0.1 nanometers to about 100 microns, and in some embodiments, from about 1 nanometer to about 10 microns. Commercially available examples of suitable particles include fluorescent carboxylated microspheres sold by Molecular Probes, Inc. under the trade names “FluoSphere” (Red 580/605) and “TransfluoSphere” (543/620), as well as “Texas Red” and 5- and 6-carboxytetramethylrhodamine, which are also sold by Molecular Probes, Inc. In addition, commercially available examples of suitable colored, latex microparticles include carboxylated latex beads sold by Bang's Laboratory, Inc.

The location of the control zone 32 may vary based on the nature of the test being performed. In the illustrated embodiment, for example, the control zone 32 is defined by the chromatographic medium 23 and positioned downstream from the detection zone 31. In such embodiments, the control zone 32 may contain a material that is non-diffusively immobilized and forms a chemical and/or physical bond with the control reagents. When the control reagents contain latex particles, for instance, the control zone 32 may include a polyelectrolyte that binds to the particles. Various polyelectrolytic binding systems are described, for instance, in U.S. Patent App. Publication No. 2003/0124739 to Song, et al., which is incorporated herein in it entirety by reference thereto for all purposes. In alternative embodiments, however, the control zone 32 may simply be defined by a region of the absorbent material 28 to which the control reagents flow after traversing through the chromatographic medium 23.

Regardless of the particular control technique selected, the application of a sufficient volume of the test sample to the device 20 will cause a signal to form within the control zone 32, whether or not the analyte is present. Among the benefits provided by such a control zone is that the user is informed that a sufficient volume of test sample has been added without requiring careful measurement or calculation. This provides the ability to use the lateral flow device 20 without the need for externally controlling the reaction time, test sample volume, etc.

The sample application zone 24, reagent zone 22, detection zone 31, control zone 32, and any other zone employed in the lateral flow device 20 may generally provide any number of distinct detection regions so that a user may better determine the concentration of the enzyme within the test sample. Each region may contain the same or different materials. For example, the zones may include two or more distinct regions (e.g., lines, dots, etc.). The regions may be disposed in the form of lines in a direction that is substantially perpendicular to the flow of the test sample through the device 20. Likewise, in some embodiments, the regions may be disposed in the form of lines in a direction that is substantially parallel to the flow of the test sample through the device 20.

One particular embodiment of a method for detecting the presence of glucose within a test sample using the device 20 of FIG. 1 will now be described in more detail. Initially, a test sample containing glucose is applied to the sample application zone 24 and travels in the direction “L” to the reagent zone 22. At the reagent zone 22, glucose mixes with glucose oxidase to initiate the catalytic reaction and generate hydrogen peroxide. Hydrogen peroxide, in turn, reacts with iodide ions contained with the reagent zone 22 to provide an intermediate compound with a high oxidation potential. Optionally, a color developer (e.g., starch) may also be employed within the reagent zone 22 to enhance the resulting color change. The desired reactions may occur while at the reagent zone 22 or as the mixture flows through the device 20. Regardless, the test sample containing the oxidative intermediate compound eventually flows to the detection zone 31, where it reacts with a chromogen in the presence of an anionic compound.

After the reaction, the chromogen changes color. The degree to which the chromogen changes color may be determined either visually or using instrumentation. In one embodiment, color intensity is measured with an optical reader. The actual configuration and structure of the optical reader may generally vary as is readily understood by those skilled in the art. Typically, the optical reader contains an illumination source that is capable of emitting electromagnetic radiation and a detector that is capable of registering a signal (e.g., transmitted or reflected light). The illumination source may be any device known in the art that is capable of providing electromagnetic radiation, such as light in the visible or near-visible range (e.g., infrared or ultraviolet light). For example, suitable illumination sources that may be used in the present invention include, but are not limited to, light emitting diodes (LED), flashlamps, cold-cathode fluorescent lamps, electroluminescent lamps, and so forth. The illumination may be multiplexed and/or collimated. In some cases, the illumination may be pulsed to reduce any background interference. Further, illumination may be continuous or may combine continuous wave (CW) and pulsed illumination where multiple illumination beams are multiplexed (e.g., a pulsed beam is multiplexed with a CW beam), permitting signal discrimination between a signal induced by the CW source and a signal induced by the pulsed source. For example, in some embodiments, LEDs (e.g., aluminum gallium arsenide red diodes, gallium phosphide green diodes, gallium arsenide phosphide green diodes, or indium gallium nitride violet/blue/ultraviolet (UV) diodes) are used as the pulsed illumination source. One commercially available example of a suitable UV LED excitation diode suitable for use in the present invention is Model NSHU550E (Nichia Corporation), which emits 750 to 1000 microwatts of optical power at a forward current of 10 milliamps (3.5-3.9 volts) into a beam with a full-width at half maximum of 10 degrees, a peak wavelength of 370-375 nanometers, and a spectral half-width of 12 nanometers.

In some cases, the illumination source may provide diffuse illumination to the chromogen. For example, an array of multiple point light sources (e.g., LEDs) may simply be employed to provide relatively diffuse illumination. Another particularly desired illumination source that is capable of providing diffuse illumination in a relatively inexpensive manner is an electroluminescent (EL) device. An EL device is generally a capacitor structure that utilizes a luminescent material (e.g., phosphor particles) sandwiched between electrodes, at least one of which is transparent to allow light to escape. Application of a voltage across the electrodes generates a changing electric field within the luminescent material that causes it to emit light.

The detector may generally be any device known in the art that is capable of sensing a signal. For instance, the detector may be an electronic imaging detector that is configured for spatial discrimination. Some examples of such electronic imaging sensors include high speed, linear charge-coupled devices (CCD), charge-injection devices (CID), complementary-metal-oxide-semiconductor (CMOS) devices, and so forth. Such image detectors, for instance, are generally two-dimensional arrays of electronic light sensors, although linear imaging detectors (e.g., linear CCD detectors) that include a single line of detector pixels or light sensors, such as, for example, those used for scanning images, may also be used. Each array includes a set of known, unique positions that may be referred to as “addresses.” Each address in an image detector is occupied by a sensor that covers an area (e.g., an area typically shaped as a box or a rectangle). This area is generally referred to as a “pixel” or pixel area. A detector pixel, for instance, may be a CCD, CID, or a CMOS sensor, or any other device or sensor that detects or measures light. The size of detector pixels may vary widely, and may in some cases have a diameter or length as low as 0.2 micrometers.

In other embodiments, the detector may be a light sensor that lacks spatial discrimination capabilities. For instance, examples of such light sensors may include photomultiplier devices, photodiodes, such as avalanche photodiodes or silicon photodiodes, and so forth. Silicon photodiodes are sometimes advantageous in that they are inexpensive, sensitive, capable of high-speed operation (short risetime/high bandwidth), and easily integrated into most other semiconductor technology and monolithic circuitry. In addition, silicon photodiodes are physically small, which enables them to be readily incorporated into various types of detection systems. If silicon photodiodes are used, then the wavelength range of the emitted signal may be within their range of sensitivity, which is 400 to 1100 nanometers.

Optical readers may generally employ any known detection technique, including, for instance, luminescence (e.g., fluorescence, phosphorescence, etc.), absorbance (e.g., fluorescent or non-fluorescent), diffraction, etc. In one particular embodiment of the present, the optical reader measures color intensity as a function of absorbance. In one embodiment, absorbance readings are measured using a microplate reader from Dynex Technologies of Chantilly, Va. (Model # MRX). In another embodiment, absorbance readings are measured using a conventional test known as “CIELAB”, which is discussed in Pocket Guide to Digital Printing by F. Cost, Delmar Publishers, Albany, N.Y. ISBN 0-8273-7592-1 at pages 144 and 145. This method defines three variables, L*, a*, and b*, which correspond to three characteristics of a perceived color based on the opponent theory of color perception. The three variables have the following meaning:

L*=Lightness (or luminosity), ranging from 0 to 100, where 0=dark and 100=light;

a*=Red/green axis, ranging approximately from −100 to 100; positive values are reddish and negative values are greenish; and

b*=Yellow/blue axis, ranging approximately from −100 to 100; positive values are yellowish and negative values are bluish.

Because CIELAB color space is somewhat visually uniform, a single number may be calculated that represents the difference between two colors as perceived by a human. This difference is termed ΔE and calculated by taking the square root of the sum of the squares of the three differences (ΔL*, Δa*, and Δb*) between the two colors. In CIELAB color space, each ΔE unit is approximately equal to a “just noticeable” difference between two colors. CIELAB is therefore a good measure for an objective device-independent color specification system that may be used as a reference color space for the purpose of color management and expression of changes in color. Using this test, color intensities (L*, a*, and b*) may thus be measured using, for instance, a handheld spectrophotometer from Minolta Co. Ltd. of Osaka, Japan (Model # CM2600d). This instrument utilizes the D/8 geometry conforming to CIE No. 15, ISO 7724/1, ASTME1164 and JIS Z8722-1982 (diffused illumination/8-degree viewing system. The D65 light reflected by the specimen surface at an angle of 8 degrees to the normal of the surface is received by the specimen-measuring optical system. Still another suitable optical reader is the reflectance spectrophotometer described in U.S. Patent App. Pub. No. 2003/0119202 to Kaylor, et al., which is incorporated herein in its entirety by reference thereto for all purposes. Likewise, transmission-mode detection systems may also be used in the present invention.

If desired, the intensity of the color at the detection zone 31 may be measured to quantitatively or semi-quantitatively determine the level of analyte present in the test sample. The intensity of the color at the detection zone 31 is typically directly proportional to hydrogen peroxide and analyte concentration. The intensity of the detection signal “Iₛ” produced at the detection zone 31 may also be compared to a predetermined detection curve developed for a plurality of known analyte concentrations. To determine the quantity of the analyte in an unknown test sample, the signal may simply be converted to analyte concentration according to the detection curve. Regardless, the analyte and any unreacted reagents then travel past the detection zone 31 until they-reach the absorbent material 28. In some cases, the reagents will oxidize over a period of time in air to form colored compounds. However, because such colored compounds are not located at the detection region 31, they generally do not interfere with the detection accuracy.

The present invention provides a relatively simple, compact and cost-efficient device for accurately detecting the presence of certain analytes (e.g., glucose) within a test sample. The test result may be visible so that it is readily observed by the person performing the test in a prompt manner and under test conditions conducive to highly reliable and consistent test results. The test is also rapid and may be detected within a relatively short period of time. For example, the chromogen may undergo a detectable color change in less than about 30 minutes, in some embodiments less than about 10 minutes, in some embodiments less than about 5 minutes, in some embodiments less than about 3 minutes, in some embodiments less than about 1 minute, and in some embodiments, less than about 30 seconds. In this manner, the chromogen may provide a “real-time” indication of the presence or absence of the analyte.

The present invention may be better understood with reference to the following examples.

### EXAMPLE 1

A solution of leucomalachite green (“LMG”) in HCl acidified water (20 milligrams per milliliter) was made. The resulting solution was a very pale green, presumably due to some slight atmospheric oxidation of the material. A glass capillary was used to place a small amount of the solution onto a nitrocellulose membrane/half-stick lateral flow assay. The device was then placed into a freshly made concentrated solution of potassium iodide and hydrogen peroxide. The KI/peroxide solution immediately turned yellow (from production of I₂) and as the solution wicked up the membrane, the leucomalachite green on the membrane rapidly turned to a bright emerald green color. However, the newly formed green material moved up the membrane with the aqueous solution.

### EXAMPLE 2

A dilute solution of a highly substituted moderate molecular weight carboxymethylcelluose (“CMC”) in water was made. Upon addition of the solution to an acidic solution of malachite green, no precipitate was observed. Without intending to be limited by theory, the lack of a precipitate may be a result of the presence of other carboxyl groups on the polymer to allow the dye/CMC precipitate to remain soluble. The dye solution was then added to a test strip as described in Example 1. No dye capture was observed.

### EXAMPLE 3

A dilute solution of a sodium oleate in water was made. Upon addition of the solution to an acidic solution of malachite green, no precipitate was observed.

### EXAMPLE 4

A dilute solution of a 5-Sulfosalicylic acid dihydrate (5-SSA) in water was made. Upon addition of the solution to an acidic solution of malachite green, a precipitate was observed. To further test this “tight ion pair”, a small amount (approximately 20 microliters) of an acidic solution of leucomalachite green (20 milligrams per milliliter) was added to the concentrated 5-SSA solution. The resulting solution was applied to a nitrocellulose lateral flow strip via glass capillary as described in Example 1. The applied spots rapidly became a bright green and remained in place on the membrane.

### EXAMPLE 5

A test was performed as described in Example 4, except that leucocrystal violet was employed instead of leucomalachite green. Upon testing, the applied spots changed from a very pale reddish to a purple and remained in the place on the membrane.

### EXAMPLE 6

Experiments were carried out to demonstrate sensitivity to peroxide as well as required KI concentrations. Specifically, a 1-microliter droplet of a solution of 3 molar 5-sulfosalicylic acid and 0.5 millimolar leucomalachite green was applied to a nitrocellulose membrane (Millipore part no. CFSP 203000) and allowed to dry. Four wells-in a 96 well plate were filled with 190 microliters of 1.0 molar potassium iodide. To each well was added 10 microliters of solutions ranging from 0.0 wt. % (control), 0.003 wt. %, 0.03 wt. %, and 0.3 wt. % hydrogen peroxide. The half-stick membranes were then placed in a respective well. The control strip produced a slight green spot, which is believed to be due to the oxidation of potassium iodide in air upon atmospheric standing. A color developed in each of the remaining test strips that demonstrated that the peroxide was activated by the presence of potassium oxide.

### EXAMPLE 7

An acidic solution of leucomalachite green (using 0.5 millimolar leucomalachite Green (“LMG”) and 2.0 molar sulfuric acid) was applied to a nitrocellulose membrane as described in Example 6. Although the spot did not completely dry, the resulting material was held in place.

### EXAMPLE 8

Solutions were made from saturated sodium sulfate and 0.5 millimolar leucomalachite green (“LMG”) and applied to a nitrocellulose membrane as described in Example 6. The solutions were developed using 180 microliters of 0.1 molar potassium iodide and 20 microliters of 3 wt. % peroxide. Although the dye remained in place, it did not readily wet the membrane.

### EXAMPLE 9

A nitrocellulose membrane was spotted with a solution of 20 mg/mL of leucomalachite green (“LMG”) dissolved in concentrated HCl (37 wt. %), diluted in a 9:1 ratio. Thereafter, 8.2 microliters of this solution was diluted to 1 milliliter with water and applied to the membrane with a capillary. Although the spot remained fixed on the membrane, it was somewhat difficult to wet the membrane. Any wetting issues were resolved by further diluting the solution to a ratio of 9:1.

### EXAMPLE 10

A solution of 10 mg/mL of leucomalachite green (“LMG”) was dissolved in concentrated HCl (37 wt. %), diluted in a 9:1 ratio. 8.2 microliters of this solution was diluted to 1 milliliter with water and applied to a nitrocellulose membrane with a capillary. When exposed to the peroxide/KI combination, the solvent in some samples did not readily pass up the membrane.

### EXAMPLE 11

A solution of 10 mg/mL of leucomalachite green (“LMG”) was dissolved in concentrated HCl (1 wt. %), diluted in a 9:1 ratio. 16.4 microliters of this solution was diluted to 1 milliliter with water and sterile filtered. The solution was then applied to a nitrocellulose membrane with a BioMec sprayer to form a continuous line across the membrane. The solution was sprayed onto the membrane at a rate of 1 microliter per centimeter and 2 microliters per centimeter. Both striped membranes showed no movement of the LMG. Dilution studies were carried out to determine the sensitivity of this assay to peroxide concentrations. All tests were run using 0.1 molar potassium iodide and a total volume of 150 or 50 microliters, depending upon the amount of LMG sprayed onto the membrane. Upon testing, the presence of hydrogen peroxide was detected in very low amounts (e.g., picomoles).

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A diagnostic test kit for detecting hydrogen peroxide released by enzyme-catalyzed oxidation of an analyte, the test kit comprising a lateral flow device that contains a chromatographic medium, wherein the chromatographic medium defines a detection zone within which a chromogen is substantially non-diffusively immobilized in conjunction with an anionic compound, the chromogen being a leuco base or a derivative thereof that is capable of undergoing a detectable color change upon oxidation.

2. The diagnostic test kit of claim 1, wherein the chromogen is a triarylmethane.

3. The diagnostic test kit of claim 2, wherein the chromogen is selected from the group consisting of leucomalachite green, pararosaniline base, crystal violet lactone, crystal violet leuco, crystal violet, Cl Basic Violet 1, Cl Basic Violet 2, Cl Basic Blue, Cl Victoria Blue, N-benzoyl leuco-methylene, and derivatives thereof.

4. The diagnostic test kit of claim 2, wherein the chromogen is leucomalachite green or a derivative thereof.

5. The diagnostic test kit of claim 1, wherein the chromogen is a diarylmethane.

6. The diagnostic test kit of claim 1, further comprising an electron donor that is capable of reacting with the hydrogen peroxide to form an intermediate compound, the intermediate compound being configured to oxidize the chromogen.

7. The diagnostic test kit of claim 6, wherein the electron donor is a source of iodide ions.

8. The diagnostic test kit of claim 7, wherein the source of iodide ions is an alkali metal iodide salt.

9. The diagnostic test kit of claim 6, wherein the electron donor is a metal.

10. The diagnostic test kit of claim 6, further comprising a color developer that is capable of complexing with the electron donor.

11. The diagnostic test kit of claim 10, wherein the color developer is a starch.

12. The diagnostic test kit of claim 1, wherein the chromogen precipitates upon oxidation.

13. The diagnostic test kit of claim 1, wherein the anionic compound is an acid, an anhydride of an acid, a salt of an acid, or a combination thereof.

14. The diagnostic test kit of claim 13, wherein the anionic compound is an inorganic acid.

15. The diagnostic test kit of claim 1, further comprising an absorbent material that receives the test sample after flowing through the chromatographic medium.

16. The diagnostic test kit of claim 1, wherein the chromatographic medium is a porous membrane.

17. The diagnostic test kit of claim 1, wherein the lateral flow assay device further comprises a reagent zone within which one or more reagents are disposed.

18. The diagnostic test kit of claim 17, wherein the reagents are selected from the group consisting of enzymes, electron donors, color developers, and combinations thereof.

19. A method for detecting hydrogen peroxide released by enzyme-catalyzed oxidation of an analyte, the method comprising:
providing a lateral flow device that comprises a chromatographic medium, the chromatographic medium defining a detection zone within which an oxidizable chromogen is substantially non-diffusively immobilized in conjunction with an anionic compound, wherein the oxidizable chromogen is a leuco base or a derivative thereof;
reacting the hydrogen peroxide with an electron donor to form an intermediate compound;
allowing the intermediate compound to flow through the chromatographic medium and contact the detection zone, whereby the intermediate compound oxidizes the chromogen and induces a color change, the oxidized chromogen having a color that differs from the color of the oxidizable chromogen; and
detecting the color of the oxidized chromogen.

20. The method of claim 19, wherein the oxidizable chromogen is a triarylmethane.

21. The method of claim 19, wherein the oxidizable chromogen is leucomalachite green or a derivative thereof.

22. The method of claim 19, wherein the electron donor is a source of iodide ions.

23. The method of claim 22, wherein the source of iodide ions is an alkali metal iodide salt.

24. The method of claim 19, wherein the electron donor is a metal.

25. The method of claim 19, further comprising a color developer that complexes with the electron donor.

26. The method of claim 25, wherein the color developer is a starch.

27. The method of claim 19, wherein the anionic compound is an acid, an anhydride of an acid, a salt of an acid, or a combination thereof.

28. The method of claim 27, wherein the anionic compound is an inorganic acid.

29. The method of claim 19, wherein the oxidizable chromogen is colorless.

30. The method of claim 19, wherein the oxidized chromogen forms a precipitate.

31. The method of claim 19, wherein the color of the oxidized chromogen is visually detected.

32. The method of claim 19, wherein the intensity of the color of the oxidized chromogen is quantitatively or semi-quantitatively measured.
